(19) United States
(12) Patent Application Publication
(10) Pub. No.: US 2009/0239303 A1
(43) Pub. Date: Sep. 24, 2009

(54) Ultrasonic Machining Fabrication of Guided Tissue Generation Surfaces and Tissue Scaffolds

(76) Inventors: Lawrence J. Rhoades, Naples, FL (US); James Randall Gilmore, Ligonier, PA (US)

Correspondence Address: IP & INTERNET LAW NORTH, LLC, P.O. BOX 38, ZELIENOPLE, PA, 16063, US

(21) Appl. No.: 12/297,344

(22) PCT Filed: Apr. 24, 2006
(86) PCT No.: PCT/US06/15483

§ 371 Date: Dec. 1, 2008

(51) Int. Cl. C12N 5/00; B24B 1/00

(52) U.S. Cl. 435/402; 451/28; 451/36

(57) ABSTRACT

Ultrasonic machining is used to fabricate at least a portion of patterned surfaces that are used directly or indirectly for guided tissue generation. Tissues may be cultivated directly in the patterned surfaces or the patterned surfaces may be used as molds for polymer tissue scaffolds.

### BACKGROUND OF THE INVENTION

1. Field of the Invention

The present invention relates to the field of tissue engineering. More specifically, the present invention relates to methods of fabricating surfaces and polymer tissue scaffolds for use in creating artificial tissues and organs.

2. Description of the Related Art

Tissue engineering involves the use of living cells as engineering materials in the quest to replicate tissue for use in the human body and other mammals. Envisioned uses of artificial tissue range from artificial skin to cartilage, to bone, and, more recently, to the development of replacement organs.

One particularly promising approach involves the generation of tissue, either directly or indirectly, upon the surfaces of silicon wafers or other substrates that have been provided with patterns for guiding tissue generation. Such patterns may include, for example, vascularization networks comprising fluidic chambers and passageways modeled after blood vessels or repositories and microchannels for functional (parenchymal) cells, neural enervation, and/or excretory systems. When used directly, cell growth is engendered upon the patterned surfaces and the resulting tissue is eventually lifted directly therefrom. When used indirectly, the patterned surface is used as a molding template onto which a polymeric material is applied to form a replica that in turn is used as a tissue scaffold into which cells will be introduced and nurtured to form a layer of artificial tissue. The details of such techniques are disclosed in United States Patent Application Publications US 2006/0019326 A1 and US 2005/0202557 A1, for example.

Typically, the patterns are provided onto or into the substrate surfaces by microfabrication processes such as photolithography; laser, plasma, or chemical etching; chemical or physical vapor deposition; electroplating; electroless plating; ion implantation; surface oxidation; and combinations thereof. Details of such techniques are described, for example, in U.S. Pat. No. 6,455,311 and Patent Cooperation Treaty International Publication No. WO 2004/026115 A2. However, the methods that have been used until now all require carefully controlled environmental and/or chemical conditions in order to be accomplished. Moreover, some of the methods, e.g., the ones that employ etching of the substrate surface, have limitations that may result in less than optimal channel cross sectional shapes and abrupt steps where channels branch out or in from one size to another. Abrupt changes in shape or depth in channels that ultimately become conduits for bodily fluids may result in dead spots and eddies which can, in turn, become the locus of infections. What is needed is a method for fabricating patterned substrate surfaces that overcome the drawbacks of the prior art.

### SUMMARY OF THE INVENTION

The present invention provides methods for fabricating patterns into substrate surfaces that can be used, either directly or indirectly, for guided tissue generation. The methods of the present invention accomplish the fabrication through the use of high precision ultrasonic machining of the patterns into the substrate surfaces. In the case where a substrate surface is to be used directly for guided tissue generation, the pattern that is ultrasonically machined into the substrate surface is a positive image of the desired pattern. In the case where the substrate surface is to be used indirectly for guided tissue generation, i.e., the substrate surface is to be used as a replica mold for a polymer tissue scaffold, the pattern that is ultrasonically machined into the substrate surface is a negative image of the desired pattern.

In some embodiments of the present invention, a portion of the guided tissue generation pattern is ultrasonically machined into the substrate surface and the balance of the pattern is micromachined into the substrate surface by one or more other microfabrication techniques such as photolithography; laser, plasma, or chemical etching; ion implantation; surface oxidation; and combinations thereof.

The present invention also includes embodiments which result in the formation of a tissue from the patterned substrate surface. These embodiments include the steps of ultrasonic machining at least a portion of the tissue generation pattern into the substrate surface; seeding cells into the patterned surface; nurturing the seeded cells to form the tissue; and removing the tissue from the patterned surface.

The present invention also includes embodiments which result in the creation of a polymer tissue scaffold. These embodiments include the steps of ultrasonic machining at least a portion of the tissue generation pattern into the substrate surface; providing a formable polymer substance; and forming a replica of at least a portion of the patterned surface with the polymer substance.

### BRIEF DESCRIPTION OF THE DRAWINGS

The criticality of the features and merits of the present invention will be better understood by reference to the attached drawings. It is to be understood, however, that the drawings are designed for the purpose of illustration only and not as a definition of the limits of the present invention.

FIG. 1 is a schematic drawing of an ultrasonic machining system usable with embodiments of the present invention.

FIG. 2. shows a schematic drawing of the work zone of the ultrasonic machining system depicted in FIG. 1.

FIG. 3 shows a plane view of a depiction of a pattern usable with embodiments of the present invention.

### DESCRIPTION OF PREFERRED EMBODIMENTS

In this section, some preferred embodiments of the present invention are described in detail sufficient for one skilled in the art to practice the present invention. It is to be understood, however, that the fact that a limited number of preferred embodiments are described herein does not in any way limit the scope of the present invention as set forth in the appended claims.

The present invention employs ultrasonic machining to fabricate patterns into substrate surfaces that can be used, either directly or indirectly, for guided tissue generation. Ultrasonic machining is a non-thermal, non-chemical process that creates no change in the microstructure, chemical or physical properties of the workpiece and results in virtually stress-free machined surfaces. The ultrasonic machining accomplishes material removal by the abrading action of an abrasive grit. Typically, the abrasive grit is introduced in slurry form between the substrate surface and the work surface of a tool that is vibrating at an ultrasonic frequency, but with small amplitude. The tool is referred to as a sonotrode. The work surface of the sonotrode itself does not directly abrade the substrate surface when the abrasive grit is added in slurry form. Rather, the vibrating sonotrode accelerates the abrasive grit particles toward and/or compresses, or hammers, them into the substrate surface, thereby causing them to gently and uniformly wear away the substrate surface material. In some applications, however, the abrasive grit, e.g., diamond particles, is fixed to the work surface of the sonotrode, rather than introduced in slurry form, a flushing liquid is flowed into the work zone to remove debris from the machining operation. Minor contributions to the material removal may come from effects of the flushing liquid or the liquid portion of the slurry in the form of cavitation-induced erosion and chemical erosion. The overall result is a precise reverse form of the shape of the working surface of the sonotrode being cut into the substrate surface.

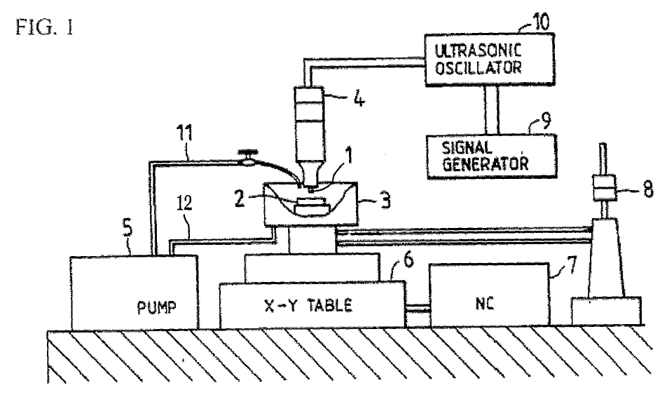

FIG. 1 presents a schematic of an exemplar conventional ultrasonic machining system that may be used in practicing the present invention. In the system shown, the sonotrode 1 and the workpiece 2 are pushed together by way of the hydraulic force applied to the machining stand 3 (shown partially cutaway) by hydraulic device 8. The workpiece 2 and the sonotrode 1 are relatively positionable in the horizontal plane by way of the X-Y table 6 which is controlled by a numerical control (NC) device 7. A signal generator 9 and an ultrasonic oscillator 10 operate in combination to cause an ultrasonic vibrator 4 to vibrate the sonotrode 1 perpendicular to its worksurface, typically with a frequency of about 20,000 cycles per second (20 kHz). A pump 5 causes a stream of an abrasive-grit loaded slurry to flow through supply line 11 into the space between the vibrating sonotrode 1 and the workpiece 2. In addition to providing abrasive grit, the slurry also cools the sonotrode 1 and workpiece 2 surfaces and removes particles and debris from the work zone. The spent slurry is collected in the basin formed by machining stand 3 and recycled to the pump 5 through the return line 12.

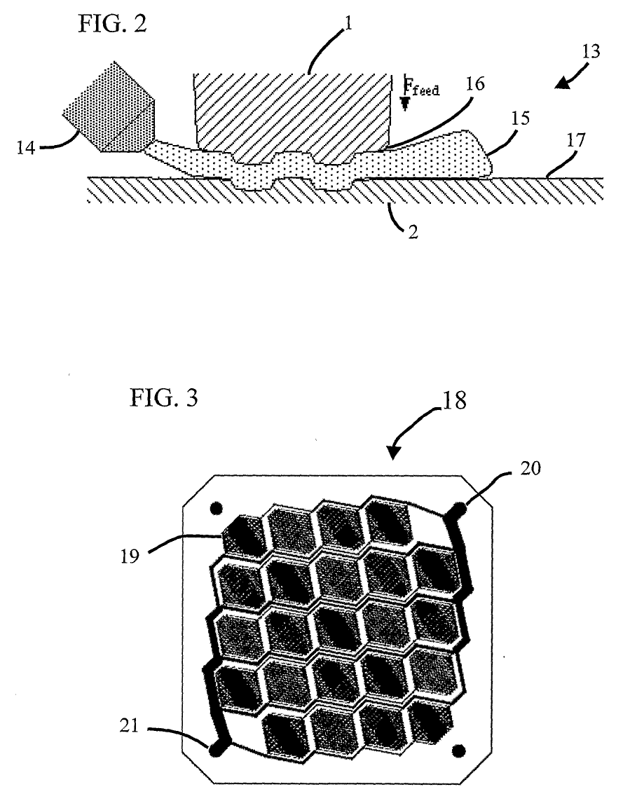

Referring now to FIG. 2, there is shown a schematic illustration of the work zone of the ultrasonic machining system depicted in FIG. 1. In the work zone 13, nozzle 14 delivers an abrasive-grit loaded slurry 15 into the gap between the work surface 16 of the sonotrode 1 and the surface 17 of workpiece 2. As the ultrasonic machining progresses, the sonotrode 1 is fed into the workpiece 2 with a predetermined force and a precise reverse form of the pattern on the worksurface 16 of the sonotrode 1 is machined into the surface 17 of the workpiece 2.

In the present invention, the substrates that are to be used directly or indirectly for guided tissue generation are the workpieces for the ultrasonic machining process. In order to be useable with the present invention, the substrate material must be amenable to ultrasonic machining. Persons skilled in the art of ultrasonic machining will recognize that materials such ceramics, glass, semiconductors, and hard and/or brittle metals and alloys are amenable to ultrasonic machining, while softer materials generally are not. Another factor to be taken into consideration when selecting a substrate material for use with the present invention is whether the patterned substrate surface is to be used directly or is it to be used indirectly. For substrates that are to be used directly, the substrate material needs to either be compatible with the cells, nutrients, waste products, and other materials that are attendant to tissue growth or be able to be coated with an interface material that has the requisite compatibility. For substrates that are to be used indirectly, the substrate needs to be compatible with the formable polymer materials that will be used to make the tissue scaffold. Silicon is a particularly preferred substrate material for use with the present invention, and it may be used as a patterned substrate that is usable either directly or indirectly. Another particularly preferred substrate material is graphite, especially graphite that has a grain size of less than one micrometer. Other preferred materials include borosilicate glasses (especially PYREX® glass, available from Corning, Corning, N.Y., US), ceramic materials, hydroxyapatite, calcium carbonate, silicon dioxide, stainless steel, titanium alloys, nickel alloys, and gold alloys.

The sonotrode material which will be used for ultrasonic machining at least a portion of the pattern into the substrate surface may be any material that is suitable for use as a sonotrode for the particular substrate and abrasive grit with which it is to be used in combination in practicing the present invention. Particularly preferred sonotrode materials for use with the present invention are aluminum alloys, titanium alloys, carbon steels, stainless steels, and tool steels. Among the more preferred tool steels are grades A2, D2, O2, and grades of the M-series.

The present invention contemplates that the pattern may be machined into the sonotrode material by any means or combination of means known to one skilled in the art for machining sonotrode work surfaces. Preferably, the pattern is machined into the work surface of the sonotrode by milling, grinding, and/or electrical discharge machining (EDM). EDM is particularly preferred when the sonotrode material has been hardened or when it will contain intricate female features that are not possible to directly machine by other machining processes. Where EDM is used, it is preferred that the EDM electrode is chosen to be either copper or graphite. In cases where graphite is used as the EDM electrode material and the working surface of the sonotrode is to make feature sizes of about 50 micrometers or less, it is particularly preferred that the graphite be of a grade that has a grain size of less than one micrometer.

In embodiments of the present invention in which the resulting patterned surface is to be used directly for the formation of tissue, the work surface of the sonotrode is configured to have the negative image of at least a portion of the selected pattern. Conversely, in embodiments of the present invention in which the resulting patterned surface is to be used to create a polymer tissue scaffold, the work surface of the sonotrode is configured to have the positive image of at least a portion of the selected pattern. Those skilled in the art will understand that the features of the aforementioned positive and negative images on the sonotrode work surfaces have dimensions which are slightly undersized from those of the selected pattern. The amount of undersize is typically in the range of about 5 to about 50 micrometers (about 0.0002 to about 0.002 inches) per feature side. The small gap occasioned by the undersize accommodates the abrasive grit during the ultrasonic machining operation.

In practicing the present invention, the selection of the pattern for guided tissue generation is to be based upon the desired features of the tissue or the polymer tissue scaffold that is to be produced. Teachings about such patterns may be found, for example, in United States Patent Application Publication US 2006/0019326 A1. Some such patterns include a pass-through feature for permitting fluid communication perpendicular to the plane of said pattern.

An example of vascularized tissue pattern is shown in FIG. 3. Referring to FIG. 3, the pattern 18 comprises twenty-three interconnected capillary beds 19 located between an inlet 20 and an outlet 21.

The general shape of a sonotrode working surface is usually round, square, or rectangular (with an aspect ratio about 3 to 1 or less). For typical ultrasonic machining devices, the sonotrode working surface is no more than about 58 square centimeters (about 9 square inches). The present invention includes fabricating patterned substrates wherein the size of the pattern to be made is within the size limitation of a single sonotrode work surface, as well as those which exceed the size of a sonotrode work surface. In embodiments wherein the pattern size exceeds that of a sonotrode work surface, a single sonotrode may be used multiple times or multiple sonotrodes may be used wherein each sonotrode is used for making a part of the overall pattern using registration techniques known in the art to achieve alignment of each portion of the pattern.

The present invention includes embodiments wherein ultrasonic machining is used to fabricate a portion of the selected pattern into the substrate and other processes are used to fabricate the remaining portion of the selected pattern. For example, in some patterns the smallest feature size may approach or be below the lower limit that reliably may be achieved by the available ultrasonic machining device. For example, some vascularization patterns include blood vessel diameters on the order of 10 microns, a size which is below that achievable on some ultrasonic machining devices. In such situations, the present invention includes embodiments wherein ultrasonic machining is used to fabricate the features of sizes down to the reliably producible feature size of the particular device, e.g., 50 microns, and features below that size are fabricated by another process. Such other processes include, for example, photolithography; laser, plasma, or chemical etching; ion implantation; surface oxidation; and combinations thereof. However, it is to be understood that embodiments of the present invention which use ultrasonic machining in combination with other fabrication processes are not restricted to situations wherein other fabrication methods are used only to make the smallest features of the desired pattern. Rather, the present invention includes within its scope all fabrications of patterned substrates for use as tissue generation guides wherein at least a substantial portion of the pattern is fabricated by ultrasonic machining.

Conventional ultrasonic machining slurries may be used in practicing embodiments of the present invention. Preferably, the slurry comprises a mixture of water, abrasive grit, and a rust inhibitor. The liquid vehicle may be a liquid other than water, e.g., organic liquids, or a combination of liquids. Suspension agents may also be present in the slurry to help maintain the abrasive grit in suspension. The slurry may also contain components to adjust its viscosity, as higher viscosities tend to lower the metal removal rate during ultrasonic machining. The nominal particle size of the grit may be in the range of about 165 microns to about 7 microns (i.e., United States Standard Sieve sizes 80 to 1000), with the size of the grit being chosen taking into consideration the finest feature size of the pattern to be fabricated into the substrate and the desired metal removal rate during the ultrasonic machining. The finer the feature size, the finer the grit size that is desirable, but also the lower the attendant metal removal rate. The slurry typically comprises between about 20 and about 60 volume percent abrasive grit. The type of abrasive grit may be of any conventional type and is selected depending upon the sonotrode and substrate materials that are to be used. Preferably, however, the abrasive grit is silicon carbide, aluminum oxide, and, for very hard materials, either boron carbide, boron silicarbide, or diamond.

Operational conditions for conducting the ultrasonic machining according to the present invention depend, in a conventional manner, on the geometric characteristics of the selected pattern and on the materials chosen for the substrate, the sonotrode working surface, and the abrasive grit. The following are examples of typical operating conditions that may be used. A frequency may be used in the range of about 15,000 to about 40,000 cycles per second (about 15 to about 40 kHz), and more preferably between about 18,000 to about 22,000 cycles per second (about 18 to about 22 kHz), with an amplitude in the range of about 2.5 to about 100 micrometers (about 0.0001 to about 0.002 inches). The feed force is generally in the range of about 22 to about 44 newtons (about 5 to about 10 pounds) and the feed rate is generally in the range of about 0.1 to about 0.25 millimeters per minute (about 0.004 to about 0.012 inches per minute). The slurry is typically flowed into the work zone at a rate of about 1 to about 3 liters per minute (about 0.26 to about 0.8 gallons per minute).

In general, the methods encompassed by the present invention comprise the steps of selecting a tissue generation pattern that is to be machined into a substrate surface; providing a substrate having a surface into which the pattern is to be machined; and ultrasonically machining at least a portion of the tissue generation pattern into the substrate surface. Some embodiments of the present invention comprise additional steps so that the method results in the formation of a tissue. These additional steps include the steps of seeding cells into the patterned surface; nurturing the seeded cells to form a tissue; and then removing the tissue from the patterned surface. Examples of how to perform these additional steps are taught by U.S. Pat. No. 6,455,311 B1 and United States Patent Application Publication 2006/0019326 A1. Also, some embodiments of the present invention comprise additional steps so that the method results in the formation of polymer tissue scaffold. These additional steps include the steps of providing a formable polymer substance; and forming a replica of at least a portion of the patterned surface with the polymer substance. An example of a particularly preferred formable polymer substance is poly(dimethyl siloxone) (PDMS). Examples of how to perform these additional steps are taught by United States Patent Application Publication 2006/0019326 A1.

While only a few embodiments of the present invention have been shown and described, it will be obvious to those skilled in the art that many changes and modifications may be made thereunto without departing from the spirit and scope of the present invention as described in the following claims. All United States patents and United States patent Publications, and Patent Cooperation Treaty Published patent applications identified herein are incorporated herein by reference in their entireties. The terms used in the appended claims are meant to be understood in view of the teachings herein and of the meanings afforded to said terms herein. Furthermore, in the event that a claim term is expressly defined by the applicants during the prosecution of this application before a patent office, that definition is to be used in construing the claim term during all proceedings before that patent office and in the patent granted or issued on this application by that patent office and that definition also hereby is expressly incorporated herein as the applicants' definition for the claim term.

What is claimed is:

1. A method that results in a patterned surface suitable for directly or indirectly guiding tissue generation, the method comprising the steps of:
a) selecting a tissue generation pattern;
b) providing a substrate having a surface;
c) ultrasonically machining at least a portion of said tissue generation pattern into said substrate surface.

2. The method of claim 1, further comprising steps so that the method results in the formation of a tissue, said further steps including:
a) seeding cells into said patterned surface;
b) nurturing said seeded cells to form said tissue; and
c) removing said tissue from said patterned surface.

3. The method of claim 1, further comprising steps so that the method results in a polymer tissue scaffold, said further steps including:
a) providing a formable polymer substance; and
b) forming a replica of least a portion of said patterned surface with said polymer substance.

4. The method of claim 3, further comprising the step of selecting said polymer substance to be poly(dimethyl siloxaone).

5. The method of claim 1, further comprising the step of selecting said substrate from the group consisting of silicon, graphite, borosilicate glasses, ceramic materials, hydroxyapatite, calcium carbonate, silicon dioxide, stainless steel, titanium alloys, nickel alloys, and gold alloys.

6. The method of claim 1, further comprising the steps of:
a) providing a sonotrode having a working surface;
b) configuring said working surface to have either the positive image or the negative image of at least a portion of said tissue generation pattern.

7. The method of claim 6, further comprising the step of ultrasonically machining at least a portion of said substrate surface with said sonotrode.

8. The method of claim 6, further comprising the step of selecting said sonotrode to comprise at least one selected from the group consisting of tool steels, titanium alloys, and aluminum alloys.

9. The method of claim 6, further comprising the step of providing said working surface with a fixed abrasive grit.

10. The method of claim 9, further comprising the step of selecting the fixed abrasive grit to be diamond.

11. The method of claim 1, further comprising the step of controlling said step of ultrasonic machining to be done in the frequency range of about 18,000 to about 22,000 cycles per second.

12. The method of claim 1, further comprising the steps of:
a) providing a sonotrode having a working surface;
b) providing an abrasive slurry, said abrasive slurry including a carrier fluid and an abrasive grit; and
c) introducing said abrasive slurry into the gap between said working surface and said substrate surface.

13. The method of claim 12, further comprising the step of selecting said abrasive grit from the group consisting of silicon carbide, aluminum oxide, boron carbide, boron silicarbide, and diamond.

14. The method of claim 12, further comprising the step of selecting said abrasive grit to have a nominal particle size in the range of about 7 to about 165 microns.

15. The method of claim 1, further comprising the step of fabricating at least a portion of said pattern into said substrate surface by a process other than ultrasonic machining.

16. The method of claim 15, further comprising the step of selecting the non-ultrasonic machining fabricating process from the group consisting of photolithography, laser etching, plasma etching, chemical etching, ion implantation, surface oxidation, and combinations thereof.

17. The method of claim 1, further comprising the step of including in said pattern a pass-through feature for permitting fluid communication perpendicular to the plane of said pattern.
